(19) United States
(12) Patent Application Publication
(10) Pub. No.: US 2021/0158941 A1
(43) Pub. Date: May 27, 2021

(54) Treatment Tracker

(71) Applicant: John Scott Taylor, Fox Island, WA (US)

(72) Inventor: John Scott Taylor, Fox Island, WA (US)

(21) Appl. No.: 16/693,349

(22) Filed: Nov. 24, 2019

(51) Int. Cl. G16H 40/20; G16H 40/63; G16H 15/00

(52) U.S. Cl. CPC G16H 40/20; G16H 15/00; G16H 40/63

(57) ABSTRACT

A treatment tracking system may include a system and ways for an entity, such as a health service provider, to track services to patients, providing at a glance what has been done and what remains to be done for a patient, which may allow better resource usage planning.

### FIELD

This disclosure relates to treatment tracking.

### BACKGROUND

Physicians and nurses often have responsibility for multiple patients at once, for example, in a hospital emergency department or an urgent care clinic. Tracking where patients are in the process of being treated has largely been done in an ad hoc method, with little consistency between various service providers.

### SUMMARY

The following presents a simplified summary of the disclosure to provide a basic understanding to the reader. This summary is not an extensive overview of the disclosure, nor does it identify key or critical elements of the claimed subject matter or define its scope. Its sole purpose is to present some concepts disclosed in a simplified form as a precursor to the more detailed description that is later presented.

The instant application discloses, among other things, a treatment tracking system, which may include a way for any entity, such as an emergency department, a veterinary office, an urgent care clinic, an inpatient department, or any other similar group, to provide visual status on what has been done and what remains to be done for a patient. For example, a display could indicate what medicine a patient is to be given, along with what has already been dispensed and what should be given next, as well as other tasks that are in process or have been completed, including lab testing, radiology procedures, or splinting. In general, a display may indicate the clinical tasks yet to be completed, for example, medications, radiology studies, consultations, or other tasks, and display the items in progress, as well as those tasks completed. The display may be customized to show details for each of the patients the user of the screen is responsible for; each nurse may see the patients for whom they are responsible, each doctor may see their patients, and administrators may see a higher-level roll-up of the patients in a unit, for example. The display may show a dashboard view, with red, green, or yellow indicators for each task or procedure, or may have other visual indicators. For example, a gradient color may be used to show how many scheduled tasks have been completed, or how far along an expected timeline a process has progressed.

The screens may be a monitor mounted on a wall, a tablet the user carries with them, a smartphone, a smartwatch, or any other device that allows displaying such data. This may improve operations by allowing users to better schedule what needs to be done more efficiently. For example, a treatment team may be able to self-organize around multiple treatment tasks for multiple patients.

This treatment tracking approach makes the progress of patients visible for caregivers. Physicians and nurses may visualize their panel of patients, for example, in the ED or inpatient setting, and their progress of care. When delays inevitably occur, it may provide notification in a timely manner, which may allow real-time course correction and response. This may result in improvements that may include saving time, reducing delays, increasing efficiency and capacity, and reducing errors and harm.

Display of the treatment tracker may be a physical board or may be an electronic screen, for example, a television, a monitor, a smartphone, a smartwatch, or dedicated hardware. It may be hosted in any manner of formats, including, for example, a shared spreadsheet, SharePoint, on-premises software, a phone app, or cloud-based software.

A treatment tracker may display different patients based on the role of the user. For example, an emergency department (ED) physician may have 8-12 patients displayed. A hospitalist physician may have 13-20 patients. A bedside nurse in the emergency department may have 4 patients, while a floor nurse may have 5 patients listed. The charge nurse on a floor may have 20-30 patients visible. The ED charge nurse may be able to see all the ED patients. A tech or therapist, or any other caregiver may have role-appropriate views built.

Other access points for the Work-In-Progress board would be available using different devices. Data could also be viewed from the side of a desktop monitor as a narrow window to monitor progress.

Many of the attendant features may be more readily appreciated as they become better understood by reference to the following detailed description considered in connection with the attached drawings.

### BRIEF DESCRIPTION OF THE DRAWINGS

FIG. 1 illustrates a user interface layout for a treatment tracker application, according to one implementation.

FIG. 2 illustrates a user interface layout for a treatment tracker.

FIG. 3 illustrates a user interface layout for a treatment tracker, according to another implementation.

FIG. 4 is a block diagram illustrating an example of a system capable of supporting a treatment tracker application, according to one embodiment.

FIG. 5 is a component diagram of a computing device to which a treatment tracker application may be applied, according to one embodiment.

### DETAILED DESCRIPTION

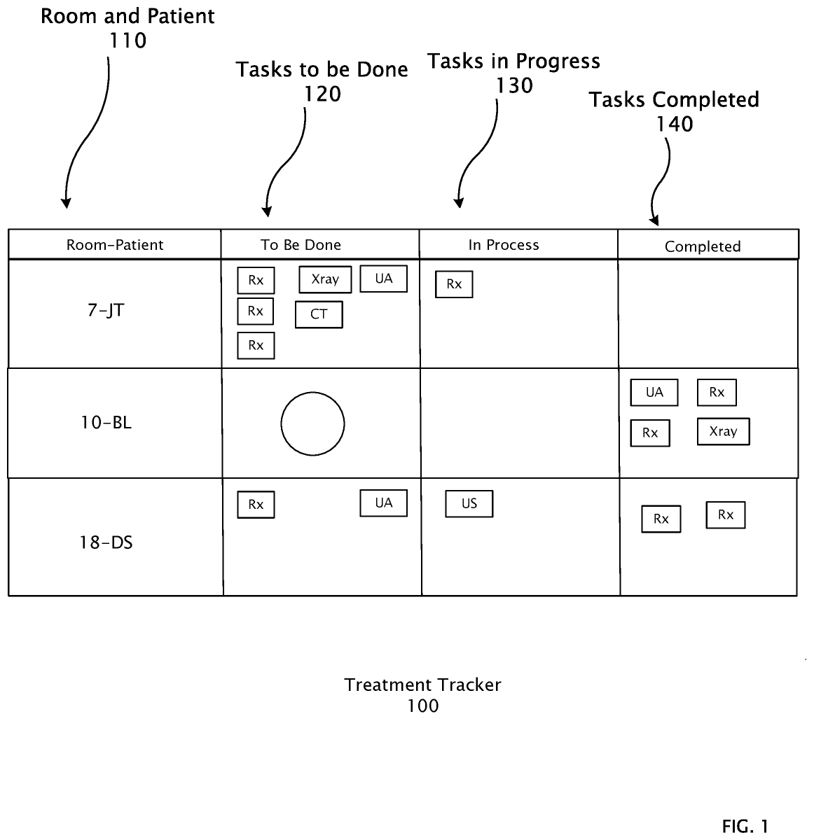

FIG. 1 illustrates a user interface layout for Treatment Tracker 100, according to one implementation. This example shows one possible view for a nurse or other caregiver. In this example, Room and Patient 110 may show each room and patient a particular caregiver is responsible for. Tasks to be Done 120 may display all tasks that need to be done for each patient. For example, an Rx task may be to give a medication, an Xray task may be to take a patient to get an X-Ray, a CT task may be to take a patient to get a CAT Scan, and a UA task may be to get a urine analysis done. Tasks in Progress 130 may show tasks a caregiver is currently working on, while Tasks Completed 140 may show tasks that have been completed for each patient.

Tasks may be identified in various ways. Tasks may be color-coded by type; for example, procedures may be orange, medications may blue. In another implementation, tasks may be color-coded by status, with Tasks Completed 140 being green, while Tasks in Progress 130 being yellow, for example. One having skill in the art will recognize that many different properties may be used to distinguish tasks, for example, size, shape, location, or other visual, audible, or tactile properties.

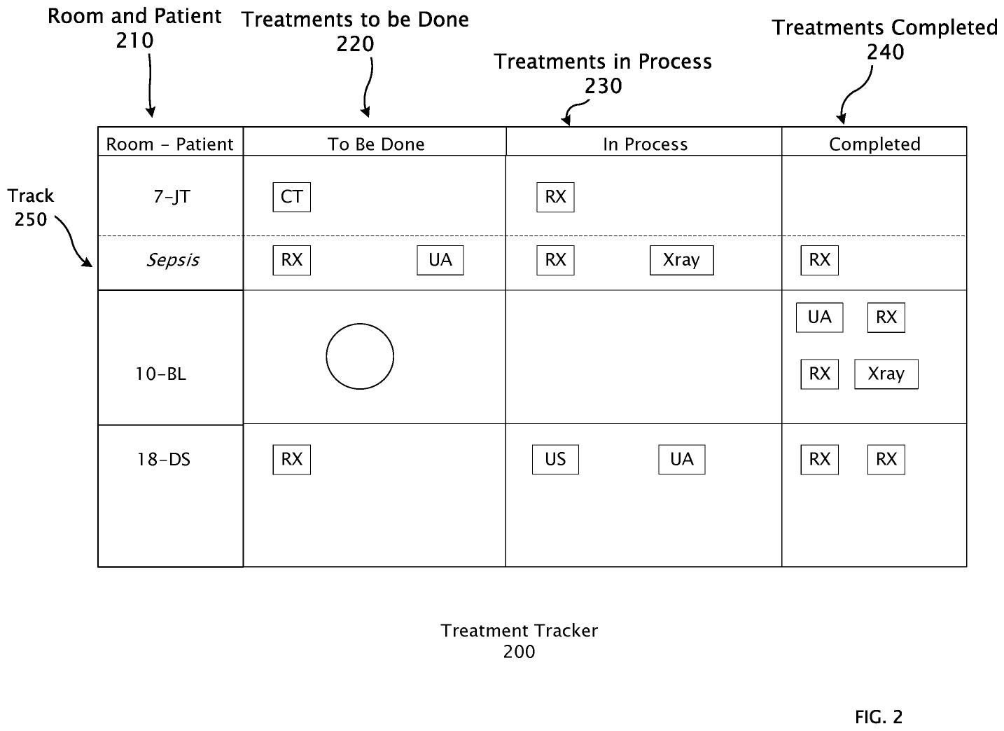

FIG. 2 illustrates a user interface layout for Treatment Tracker 200, according to another implementation. This example shows one possible view for a nurse or other caregiver. In this example, Track 250 may indicate that under Room and Patient 210, patient JT in room 7 has been diagnosed with sepsis. As a result, Treatments to be Done 220, Treatments in Process 230, and Treatments Completed 240 may have a row for general tasks for a patient, and a row for tasks related to certain diagnoses or treatments. In this example, the sepsis diagnosis indicates tasks for three medications, a urine analysis, and an X-Ray. A task for a particular diagnosis may have strict deadlines for completion, so that task may have a property indicating when it must be done, the urgency of the task, or other attributes. When a diagnosis is entered, one or more tasks may be determined by the system. If a task is nearing a deadline, it may show as red, or it may flash, for example. In another implementation, an urgent task may be displayed more brightly, or other tasks may dim to emphasize it. In another implementation, a smartphone or smartwatch may vibrate. Many types of watches for various conditions may be tracked, for example, stroke, sepsis, chronic conditions, indwelling bladder catheters, or other conditions or treatments.

One having skill in the art will recognize that many different properties may be used to distinguish tasks, for example, size, shape, location, or other visual, audible, or tactile properties.

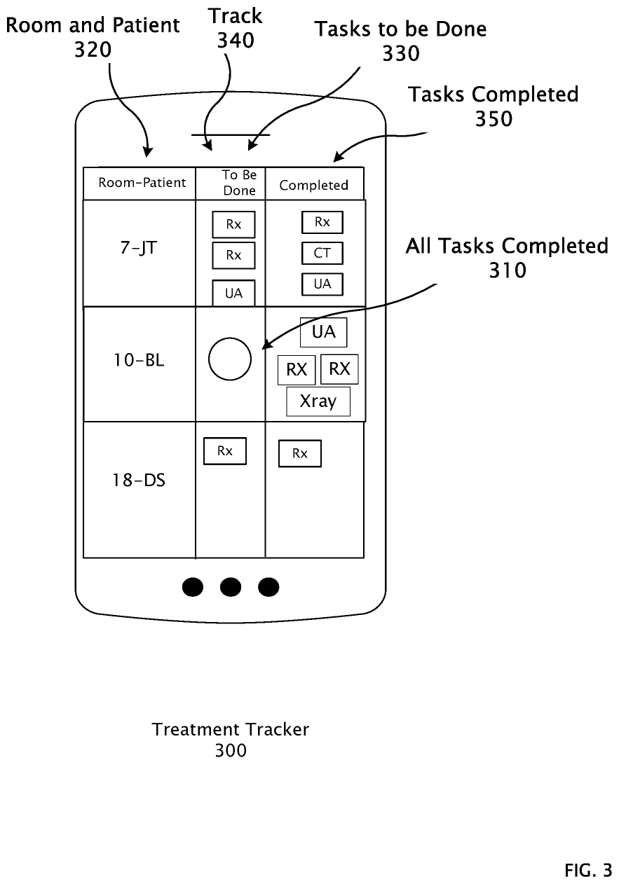

FIG. 3 illustrates a user interface layout for Treatment Tracker 300 application, according to another implementation. This example shows one possible view for a nurse or other caregiver as may be used on a smartphone or other device. In this example, Room and Patient 320 may show which patient requires a task to be done, Tasks to be Done 330 may display all tasks that remain to be done for each patient. For example, an Rx task may be to give a medication, an Xray task may be to take a patient to get an X-Ray, a CT task may be to take a patient to get a CAT Scan, and a UA task may be to get a urine analysis done. Symbol All Tasks Completed 310, such as a circle, may be used to indicate that everything related to a particular patient has been completed. Tasks Completed may show which tasks have been completed for the patient. Track 340 may indicate that certain tasks may be required because of a particular diagnosis. Tasks Completed 350 may have a row for general tasks for a patient, and a row for tasks related to certain diagnoses or treatments.

In one implementation, clicking or tapping on a task may provide additional information about the task. For example, tapping on an Rx task may show the medication to provide, while tapping on a blood work task may show which tests to run on a blood sample.

One having skill in the art will recognize that while in the example given, tasks are shown as rectangles with letters indicating a type of task, other indicators may be used. Tasks may be shown with different shapes, colors, intensity, sizes, changes in appearance such as flashing, or other visual differentiators, or audible or tactile notifications, such as a tone or vibration. Task symbols may change in appearance over time, as they become urgent. For example, a task indicator may grow in size if a deadline for completion is approaching. A task may have a timer associated with it to track when it may need to be completed. A task in progress may show a graduated color indicating how far along it is to completion. Treatment Tracker may send emails or text messages to notify one or more people of tasks status. Many types of indicators or notifications may be used to represent task status, importance, urgency, an abnormal result, comments on reasons for a delay, or any other pertinent information.

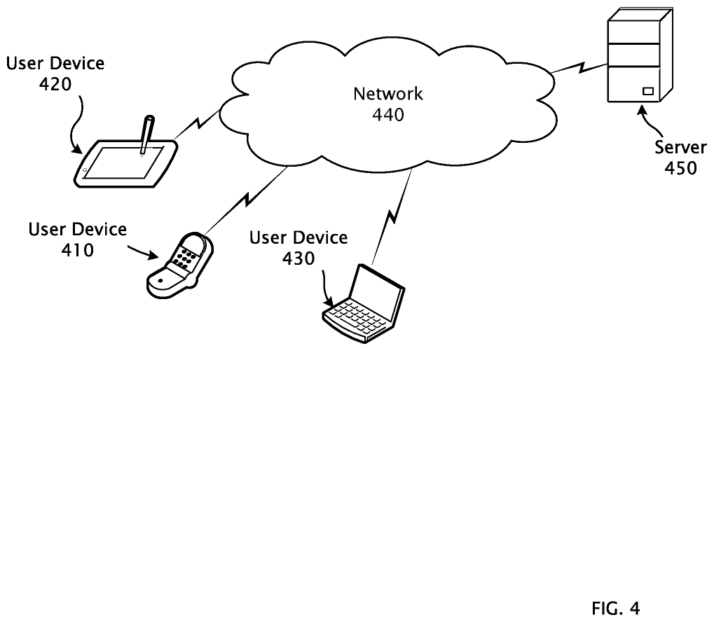

FIG. 4 is a block diagram illustrating an example of a system capable of supporting a treatment tracker, according to one embodiment. Network 440 may include Wi-Fi, cellular data access methods, such as 3G, 4G LTE, 5G, Bluetooth, Near Field Communication (NFC), the internet, local area networks, wide area networks, or any combination of these or other means of providing data transfer capabilities. In one embodiment, Network 440 may comprise Ethernet connectivity. In another embodiment, Network 440 may comprise fiber-optic connections.

User Device 410, 420, or 430 may be a smartphone, tablet, laptop computer, smartwatch, intelligent eyewear, or other devices, and may have location-based services, for example, GPS, cell phone tower triangulation capability, or accelerometers, and may have network capabilities to communicate with Server 450. Server 450 may include one or more computers and may serve a number of roles. Server 450 may be conventionally constructed, or may be of a special purpose design for processing data obtained from a treatment tracker. A provider using treatment tracker may use a server owned or managed by a service provider. One skilled in the art will recognize that Server 450 may be of many different designs and may have different capabilities. Server 450 may include one more computers and may be of a special purpose design for processing data obtained from a treatment tracker.

User Device 410, 420, or 430 may include a device application to support a treatment tracker, for example, allowing a user to choose from different views or to add tasks. In another embodiment, Device 410, 420, or 430 may display a website hosted on Server 450 in a browser, which may allow a user to request an action or input information. Server 450 may be operated by a party offering treatment tracker. Server 450 may allow a user to receive a notification of the requested action.

A using organization may also provide a User Device 410, 420, or 430 to a worker to provide access to a treatment tracker.

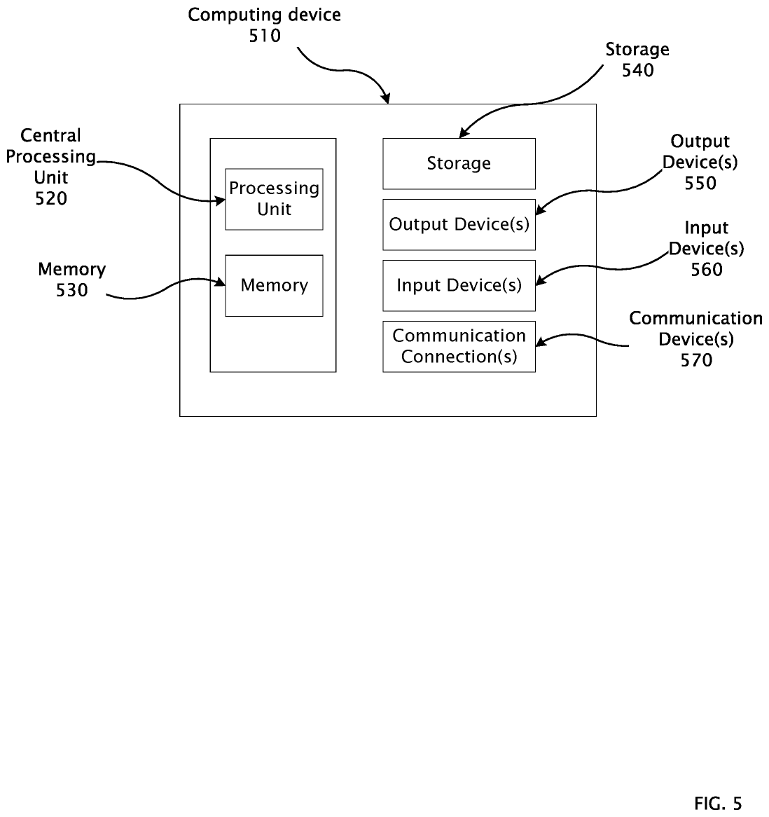

FIG. 5 is a component diagram of a computing device to which a treatment tracker may be applied, according to one embodiment. The Computing Device 510 can be utilized to implement one or more computing devices, computer processes, or software modules described herein, including, for example, a mobile device. In one example, the Computing Device 510 can be used to process calculations, execute instructions, and receive and transmit digital signals. In another example, the Computing Device 510 can be utilized to process calculations, execute instructions, receive and transmit digital signals, receive and transmit search queries and hypertext, and compile computer code suitable for a mobile device. The Computing Device 510 can be any general or special purpose computer now known or to become known capable of performing the steps and/or performing the functions described herein, either in software, hardware, firmware, or a combination thereof.

In its most basic configuration, Computing Device 510 typically includes at least one Central Processing Unit (CPU) 520 and Memory 530. Depending on the exact configuration and type of Computing Device 510, Memory 530 may be volatile (such as RAM), non-volatile (such as ROM, flash memory, etc.) or some combination of the two. Additionally, Computing Device 510 may also have additional features/functionality. For example, Computing Device 510 may include multiple CPUs. The described methods may be executed in any manner by any processing unit in Computing Device 510. For example, the described process may be executed by multiple CPUs in parallel.

Computing Device 510 may also include additional storage (removable and/or non-removable) including, for example, magnetic or optical disks or tap. Such additional storage is illustrated in FIG. 5 by Storage 540. Computer-readable storage media includes volatile and nonvolatile, removable and non-removable media implemented in any method of technology for storage of information such as computer-readable instructions, data structures, program modules, or other data. Memory 530 and Storage 540 are all examples of computer-readable storage media. Computer-readable storage media includes, but is not limited to, RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which may be used to store the desired information and which may be accessed by Computing Device 510. Any such computer-readable storage media may be part of Computing Device 510. But computer-readable storage media may be part of Computing Device 510. But computer-readable storage media does not include transient signals.

While the detailed description above has been expressed in terms of specific examples, those skilled in the art will appreciate that many other configurations could be used. Accordingly, it will be appreciated that various equivalent modifications of the above-described embodiments may be made without departing from the spirit and scope of the invention.

Additionally, the illustrated operations in the description show certain events occurring in a certain order. In alternative embodiments, certain operations may be performed in a different order, modified or removed. Moreover, steps may be added to the above-described logic and still conform to the described embodiments. Further, operations described herein may occur sequentially, or certain operations may be processed in parallel. Yet further, operations may be performed by a single processing unit or by distributed processing units.

Computing Device 510 may also contain Communication Device(s) 570 that allow the device to communicate with other devices. Communications Device(s) 570 is an example of communication media. Communication media typically embodies computer-readable instructions, data structures, program modules, or other data in a modulated data signal such as a carrier wave or other transport mechanism and includes any information delivery media. The term “modulated data signal” means a signal that has one or more of its characteristics set or changed in such a manner as to encode information in the signal. Communication media may include wired media such as a wired network or direct-wired connection, and wireless media such as acoustic, radio frequency (RF), infrared, and other wireless media. The term computer-readable media, as used herein, includes both computer-readable storage media and communication media. The described methods may be encoded in any computer-readable media in any form, such as data, computer-executable instructions, and the like.

Computing Device 510 may also have Input Device(s) 560 such as a keyboard, a mouse, a pen, a voice input device, a touch input device, etc. Output device(s) 550 such as a display, speakers, printer, etc. may also be included. All these devices are well known in the art and need not be discussed at length.

Those skilled in the art will realize that storage devices utilized to store program instructions can be distributed across a network. For example, a remote computer may store an example of the process described as software. A local or terminal computer may access the remote computer and download a part or all of the software to run the program. Alternatively, the local computer may download pieces of the software as needed, or execute some software instructions at the local terminal and some at the remote computer (or computer network). Those skilled in the art will also realize that by utilizing conventional techniques known to those skilled in the art that all, or a portion of the software instructions may be carried out by a dedicated circuit, such as a digital signal processor (DSP), programmable logic array, or the like.

While the detailed description above has been expressed in terms of specific examples, those skilled in the art will appreciate that many other configurations could be used. Accordingly, it will be appreciated that various equivalent modifications of the above-described embodiments may be made without departing from the spirit and scope of the invention.

Additionally, the illustrated operations in the description show certain events occurring in a certain order. In alternative embodiments, certain operations may be performed in a different order, modified or removed. Moreover, steps may be added to the above-described logic and still conform to the described embodiments. Further, operations described herein may occur sequentially or certain operations may be processed in parallel. Yet further, operations may be performed by a single processing unit or by distributed processing units.

The foregoing description of various embodiments of the invention has been presented for the purposes of illustration and description. It is not intended to be exhaustive or to limit the invention to the precise form disclosed. It is intended that the scope of the invention be limited not by this detailed description, but rather by the claims appended hereto. The above specification, examples, and data provide a complete description of the manufacture and use of the invention. Since many embodiments of the invention can be made without departing from the spirit and scope of the invention, the invention resides in the claims hereinafter appended.

1. A system, comprising:
a processor;
a memory, operably connected to the processor;
instructions in the memory which, when executed, perform a method, comprising:
receiving identification for a patient; and
receiving a task or diagnosis associated with the patient.

2. The system of claim 1, further comprising:
if a task is received, receiving a status of the task; and
outputting to a display an indicator of the patient and the status of the task.

3. The system of claim 1, further comprising:
if a diagnosis is received, determining one or more tasks associated with the diagnosis;
receiving at least one status of one of the one or more tasks; and
outputting to a display an indicator of the patient and the status of the task.

4. A method, comprising:
receiving identification for a patient;
receiving a task associated with the patient;
receiving a status of the task; and
outputting to a display an indicator or the patient and the status of the task.
